(19) United States
(12) Patent Application Publication
(10) Pub. No.: US 2022/0037028 A1
(43) Pub. Date: Feb. 3, 2022

(54) METHOD TO PROVIDE PERSONALIZED MEDICAL DATA

(71) Applicant: SOPHIA GENETICS S.A., Saint-Sulpice (CH)

(72) Inventors: David COX, Saint-Sulpice (CH); Gilbert PERRIN, Saint-Sulpice (CH)

(73) Assignee: SOPHIA GENETICS S.A., Saint-Sulpice (CH)

(21) Appl. No.: 17/280,330

(22) PCT Filed: Sep. 28, 2019
(86) PCT No.: PCT/EP2019/076335

§ 371 Date: Mar. 26, 2021

Related U.S. Application Data
(60) Provisional application No. 62/738,028, filed on Sep. 28, 2018.

(51) Int. Cl. G16H 50/30; G16H 10/60

(52) U.S. Cl. CPC G16H 50/30; G16H 10/60

(57) ABSTRACT

A method to provide personalized data of a patient includes obtaining at least one first personal data for a non-modifiable risk factor, obtaining at least one second personal data for a modifiable risk factor, and normalizing the first and second data using a lookup table, said normalized data representing an increase or decrease versus a neutral value. The method also includes adding the normalized data representing a decrease to a positive parameter, adding the normalized data representing an increase to a negative parameter, displaying the positive and the negative parameters in two distinct colors in a pie shape, the surface of each pie being proportional to the value of each parameter, and displaying in association with the pie shape, the portion of the negative parameter that results from the second personal data.

### INTRODUCTION

Various public health strategies have been developed worldwide to increase prevention of certain medical pathologies, such as cardiovascular disease, chronic respiratory disease, unintentional injuries, diabetes, certain infectious diseases, and cancers. A large part of the deaths caused by these diseases are indeed due to inadequate lifestyle such as poor diet or lack of exercise. In addition to awareness campaigns, early detection measures such as regular medical check-ups have also been put in place in many countries to identify any anomalies such as biomarkers as early indicators of disease onset. A major issue of this medical approach is that it is global, and does not take into account the specific parameters of the patient, such as his/her lifestyle elements as well as his/her genetic predisposition. There is therefore a need for more personalized medical advice to adapt prevention recommendations, including the need for specialized examination (screening), to the specifics of each patient. In general, the risk factors may be classified along two main categories:

1. Nonmodifiable risk factors for instance age, height, genome, family history, age of menopause onset i.e. the risks for which the patient has no control

2. Modifiable risk factors for instance alcohol intake, smoking, diet, exercise, weight, sun exposure, stress, menopausal hormone therapy intake i.e. the risk that the patient behavior can impact this risk

In the case of breast cancer prevention, it has been recently evaluated that modifiable factors significantly impact the risk for some categories of patients, as described for instance in Breast Cancer Risk From Modifiable and Nonmodifiable Risk Factors Among White Women in the United States by Maas et al. JAMA Oncol. 2016 Oct. 1; 2(10): 1295 1302. doi:10.1001/jamaonco1.2016.1025. How much risk can be decreased by modifiable factors also depends on the individual characteristics of the patient. For instance, while most countries recommend regular screening mammography for women above 50, some women may benefit from earlier screening, depending on their individual parameters for multiple factors. There is therefore a need for better determining and presenting this information in individual medical counseling. Moreover, for women with a significant contribution of modifiable factors to their personal risk assessment, there is a need for better presenting the evolution of this data in later individual medical counseling so that the benefit of e.g. lifestyle changes impacting modifiable factors can be pointed out to the patient.

### BRIEF DESCRIPTION

The proposed solution comprises two main phases, the first phase being the determination of the values and the second step being the particular display arrangement of these values in a comprehensive way.

It is proposed a method to provide personalized data of a patient comprising:
obtaining at least one first personal data for a non-modifiable risk factor,
obtaining at least one second personal data for a modifiable risk factor,
normalizing the first and second data using a lookup table, said normalized data representing an increase or decrease versus a neutral value,
adding the normalized data representing a decrease to a positive parameter,
adding the normalized data representing an increase to a negative parameter,
displaying the positive and the negative parameters in two distinct colors in a pie shape, the surface of each pie being proportional to the value of each parameter,
displaying in association with the pie shape, the portion of the negative parameter that results from the second personal data.

### BRIEF DESCRIPTION OF THE FIGURES

The present invention will be better understood with the help of the attached figures in which:

FIG. 1 illustrates a conversion table used to normalize the patient data,

FIG. 2 illustrates the first section of the display with the pie shape results,

FIG. 3 illustrates the section 1 and section 2 of the patient's result,

FIG. 4 illustrates the same as FIG. 3 for another patient.

### DETAILED DESCRIPTION

The object of the present disclosure is to provide a tool for a practitioner to present in a comprehensive way the risk situation of a patient.

The first step is to acquire the personal data for a patient. This is made usually in reference to a particular risk. By risk we understand health risk such as breast cancer, diabetes, osteoporosis, prostate cancer, etc.

For a given risk, a set of patient data is acquired and entered into the system of the present invention. For the present disclosure, we will take the example of breast cancer.

In FIG. 1, we have a table showing the influence of a given risk factor on the global risk factor. This is in the form of a lookup table with an input and an output. According to the first example of the FIG. 1, the age of the patent of her first menstruation is evaluated. On the left column, we have the age and in the right column the normalized data. In this example, it was decided that the value 1, as normalized data, is neutral, i.e. will not increase or decrease the risk. A value below 1 means that this factor reduces the global risk. Conversely, a value above 1 represents an increase risk factor.

This is more apparent in the second example for the age of the menopause. Depending of the age of the patient at menopause, the normalized data below 1 is considered positive (i.e. reducing the risk factor) or above 1 is considered negative, (i.e. increasing the risk factor).

Once all patient data are acquired and normalized, the present system calculates the current variation (CV) of the risk factor for said patient.

This is achieved by adding the natural logarithm of each normalized risk factor (nrf):

CV=ln(nrf)

The positive normalized patient data (nrf+) are added to form the positive current variation PCV=ln(nrf+) and the negative normalized patient data (nrf−) are added to from the negative current variation NCV=ln(nrf−).

Various other methods are also proposed in the frame of the present disclosure to determine the normalized risk factor. In an alternate embodiment, the FIG. 1 can contain directly the natural logarithm of the value of the tables so that the positive factors are expressed by a positive number and the negative factor are expressed by a negative number.

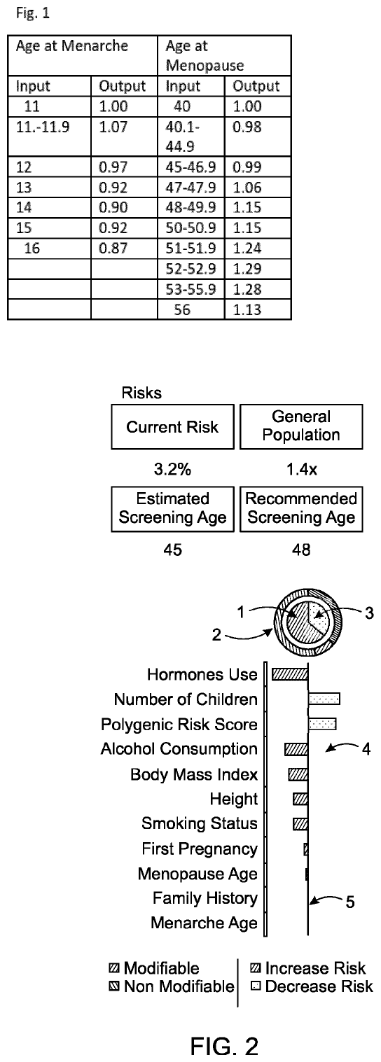

The system produces a representation of these two values in a pie shape, representing the positive (PCV) and the negative (NCV) current variation in two distinct colors. The pie is illustrated in FIG. 2. The portion 1 illustrates the increasing factors and the portion 3 illustrates the reducing factors.

The risks are organized in at least two categories, the non-modifiable risk factors and the modifiable risk factors. The non-modifiable risk factors are the factors for which the patient has no control of, for example the age of menarche. The modifiable risk factors are the factors for which the patient has an influence. This is the case for example for alcohol consumption, smoking etc.

The system then calculates the portion (PV) of the negative current variation only linked with the modifiable risk factors. The value PV represents 0 to 100% or the negative current variation.

In the FIG. 2, the section 2 represents the portion of the modifiable risk factors of the portion 1. The ring around the pie indicates the portion of the modifiable and non-modifiable risk factors. Other embodiments can be used to represent this information such as altering the texture of a part of the section 1 according to the impact of the modifiable risk factors.

The resulting image is a direct understanding of the overall risk, the repartition in positive and negative risk factors in the pie shape and the highlighting the part of the negative factors that result of modifiable risk factors. In the example of the FIG. 2, approximately 80% of the negative risk factors are the result of modifiable risk factors.

The complement the representation in pie shape, the system generates the details of the normalized value for each factor. Section 4 of FIG. 2 illustrates each factor organized around a vertical line 5. If the normalized version of the risk factor is positive (i.e. reducing the risk), the corresponding value is represented on the right side of the bar and if the normalized version of the risk factor is negative, it is represented on the life side of the bar.

According to a particular embodiment, a first color is selected for the non-modifiable risk factor and a second color is selected for the modifiable risk factor.

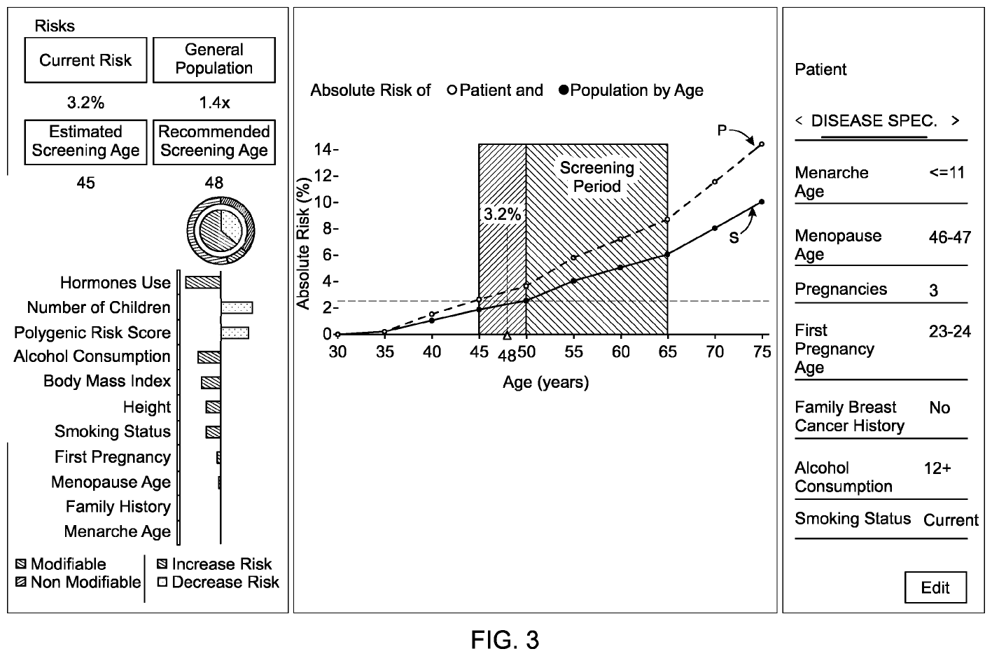

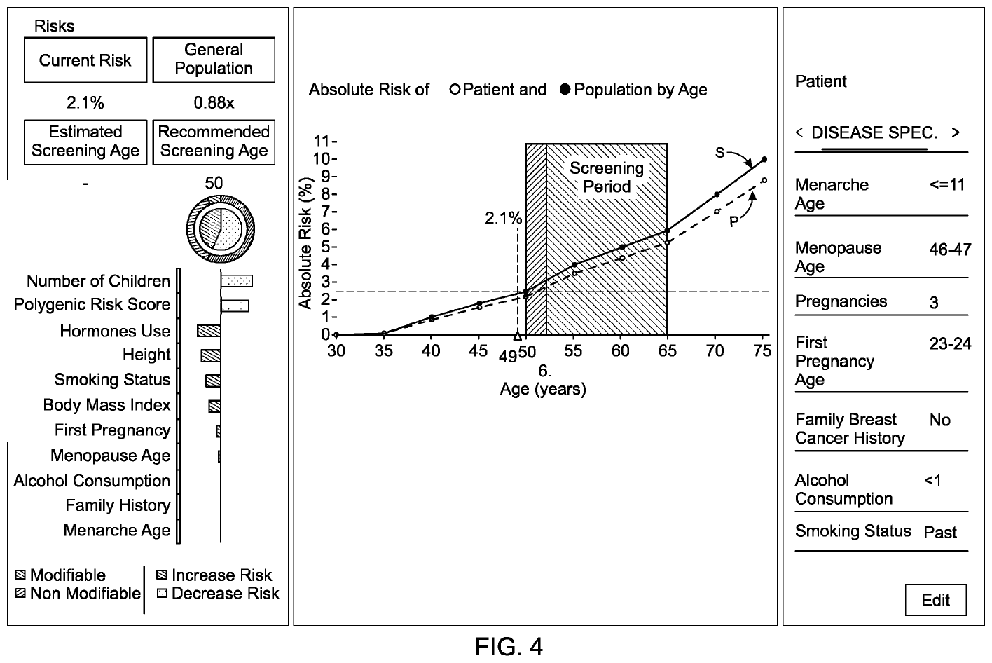

In order to determine the global risk factor, as illustrated in the left corner of the FIGS. 3 and 4, the system should determine the reference risk factor of said patient. This is executed by accessing a lookup table comprising for each age, the reference risk factor. Based on the age of the patient, the current standard risk factor is then determined.

In order to calculate the global risk factor, the reference risk factor is added to the current variation CV.

In the portion 6 of the FIG. 3 (and FIG. 4), the line P represents the standard risk factor from 30 to 75 years.

In the example of the FIG. 3, the age of the patient is 48 and the current standard risk factor (for a given population) is 2.4%. In this example the current variation for this patient represents 0.8% meaning that this patient is 0.8% higher than the standard risk. The global risk factor (or current risk as represented in the FIG. 4) is therefore 3.2%.

The system then determines the standard screening age which is dependent of the standard risk factor. For the breast cancer, it was determined that the screening has to be executed for a risk factor of 2.5% (see horizontal dotted line). This corresponds to the age of 50 in the standard population (see line S in FIGS. 3 and 4). However, at 48, this patient is already above this level and should therefore start the screening immediately.

In the example of the FIG. 4, the patient is represented by the line P which is below the standard risk for a given population (line S). The system then determines the age at which this patient will be at 2.5% and the system determine the age of 50 for the first screening (based on current medical knowledge).

The present method is executed on a system comprising at least one processing and at least one memory. The memory stores the general data common to all patients such as the lookup tables. For each specific risk, as defined above, a lookup table is stored and used to determine the risk factor related to a given patient for a given risk.

The processor receives the input data for a patient and calculates the risk factor. The processor sorts the information in order to present the result in a comprehensive way. In the example of the FIG. 2, the risk factors are presented in a list for which the first risk factor on top of the list is the risk factor having the highest absolute value. In this way, the highest value draws the attention of the operator.

The system comprises a graphical interface to prepare the data for the display.

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise specifically noted, articles depicted in the drawings are not necessarily drawn to scale.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, each refers to each member of a set or each member of a subset of a set.

1. A method to provide personalized data of a patient comprising:
obtaining at least one first personal data for a non-modifiable risk factor,
obtaining at least one second personal data for a modifiable risk factor,
normalizing the first and second data using at least a lookup table, said normalized data representing an increase or decrease versus a neutral value,
adding the normalized data representing a decrease to a positive risk factor,
adding the normalized data representing an increase to a negative risk factor,
displaying the positive and the negative risk factors in two distinct colors in a pie shape, the surface of each portion of the pie being proportional to the value of each parameter,
determining the part of the negative risk factor related to the modifiable risk factor,
displaying in association with the pie shape, said part that results from the second personal data.

2. The method of claim 1, further comprising:
displaying the first and the second normalized data personal data with a reference of the description of the first and second risk factor.

3. The method of claim 2, further comprising:
sorting the first and the second normalized data personal according to their respective absolute value, the highest absolute value being placed on top of a list,
displaying the list of normalized personal data with a reference of a description of their respective risk factor.

4. The method of claim 1, further comprising:
calculating a current variation risk factor by adding the natural logarithm of each normalized risk factor,
displaying the current risk factor.

5. The method of claim 5, further comprising:
extracting from a reference table, the reference risk factor based on one personalized data of a patient,
determining a current variation (CV) of the risk factors according to CV=Σ ln(nrf) in which each nrf is the normalized risk factor,
determining a global risk factor by adding current variation (CV) to the reference risk factor,
displaying the global risk factor.
